(19) United States
(12) Patent Application Publication
(10) Pub. No.: US 2024/0091470 A1
(43) Pub. Date: Mar. 21, 2024

(54) PNEUMATIC STIMULATION DEVICES, SYSTEMS AND METHODS

(71) Applicant: INVENT MEDICAL CORPORATION, Carlsbad, CA (US)

(72) Inventor: Samuel M. CHANG, Carlsbad, CA (US)

(21) Appl. No.: 18/369,057

(22) Filed: Sep. 15, 2023

Related U.S. Application Data
(60) Provisional application No. 63/407,301, filed on Sep. 16, 2022.
(60) Provisional application No. 63/423,197, filed on Nov. 7, 2022.

(51) Int. Cl. A61M 16/00; A61M 16/16; G16H 40/63; G16H 50/20

(52) U.S. Cl. CPC A61M 16/0069; A61M 16/0003; A61M 16/026; A61M 16/16; G16H 40/63; G16H 50/20; A61M 2016/003; A61M 2205/3303; A61M 2205/3306; A61M 2205/3327; A61M 2205/3334; A61M 2205/3344; A61M 2205/3351; A61M 2205/50; A61M 2206/20; A61M 2230/005; A61M 2230/20; A61M 2230/40

(57) ABSTRACT

A pneumatic stimulation system, comprising: a flow device; a patient interface fluidly coupled to the flow/pressure generation device via at least one conduit; a controller operably connected to the flow device, and configured to receive sensor data from at least one sensor, and to control a flow of a gas from the flow device to the patient interface based at least in part on the sensor data.

### CROSS-REFERENCE TO RELATED APPLICATIONS

This application claims priority to U.S. Provisional Patent Application Nos. 63/407,301, filed on Sep. 16, 2022, and 63/423,197, filed Nov. 7, 2022, which is hereby incorporated herein by reference as if set forth in full.

### TECHNICAL FIELD

The embodiments described herein are generally directed to pneumatic stimulation devices, systems and methods.

### BACKGROUND

One of the most common treatments for sleep apnea is positive airway pressure (PAP) treatment, including continuous (CPAP) and bi-level options. A CPAP machine takes in room air, filters and pressurizes it, and delivers it through a tube and into a mask worn by a person. The traditional CPAP and bi-level treatment mechanism is pressure. Flow rate is a means of providing the pressure. Some other exemplary flow generation systems that generate a gaseous flow, for example, airflow or a blend of ambient air and oxygen, include those described in Applicant's own U.S. 2016/0287824, U.S. Pat. Nos. 10,525,222, and 10,869,977. These and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

A widely used mouth device for mild to moderate sleep apnea is a mandibular advancement device (MAD). MADs are like mouth guards that snap over dental arches and have metal hinges that make it possible for the lower jaw to be eased forward. However, they are associated with bite changes, TMJ disorders, loose teeth, drooling, mouth dryness, and other risks.

Yet another treatment for sleep apnea is eXciteOSA, a daytime sleep apnea treatment that aims to strength tongue muscles. Some other treatments for sleep apnea involve surgery, such as uvulopalatopharyngoplasty (UPPP) and implantable devices that send electrical pulses to the hypoglossal nerve (Inspire therapy).

### SUMMARY

Systems and methods for the detection, reduction and prevention of sleep disordered breathing (e.g., obstructive sleep apnea, snoring, abnormal and/or difficult respiration) events are provided herein. Contemplated systems and methods can advantageously reduce or minimize the incidence of sleep apnea and/or incidents of SpO₂ falling below the baseline (i.e., non-obstructive state) during sleep, and/or other sleep disordered breathing. In some aspects, systems and methods are provided for stimulation of the hypoglossal nerve via a gas (e.g., oxygenated air) delivered as a pulse, a set of pulses, and/or sets of pulses via a patient interface to stimulate the hypoglossal nerve and prevent the tongue from obstructing an airway. In some aspects, the delivery mechanism can comprise a flow (or flow rate) and/or a volume. In some aspects, the control mechanism can be pressure (which could be an indirect way of producing flow rate/volume. Such devices, systems and methods can advantageous enhance patient compliance and associated clinical and quality of life benefits. Further, such devices and systems provide a simpler, lighter, inexpensive, patient-friendly obstructive sleep apnea (and/or other sleep disordered breathing) treatment.

In some aspects, a pneumatic stimulation system is provided, comprising a flow device (e.g., a flow and/or pressure generation device (a flow/pressure generation device)), a patient interface (e.g., a cannula, a nasal pillow, a mask, an oral mask) fluidly coupled to the flow/pressure generation device via at least one conduit (e.g., hoses, tubing), and a controller operably connected to the flow/pressure generation device, and configured to receive sensor data from at least one sensor, and to control a flow rate of a gas from the flow/pressure generation device to the patient interface based at least in part on the sensor data. As used herein, the term “patient interface” should be interpreted broadly to include interfaces such as cannulas, nasal pillows, masks, oral masks, nasal prongs, etc. configured to be worn by a subject receiving pneumatic stimulation as described herein. In some aspects, the sensor can be positioned within the at least one conduit (e.g., tubing). In some aspects, the sensor can be placed on a patient/user.

In some aspects, a pneumatic stimulation system is provided, comprising a flow device, a patient interface fluidly coupled to the flow device via at least one conduit, and a controller operably connected to the flow device, and configured to cause the flow device to deliver a pulse (which may also referred to herein as a bolus) of a gas (e.g., air, gas mixture) or a set/sets of pulses (which may also referred to herein as a set of boluses) of one or more gases via the patient interface. In some embodiments, the controller is configured to cause the flow device to deliver the pulse(s) of the gas based on a time interval. In some embodiments, the controller is configured to cause the flow device to deliver the pulse(s) of the gas based on a user's breathing activity (e.g., based on a breath trigger/start). In some aspects, random delivery (random in terms of pulse(s) amount, number of pulse(s), pulse(s) frequency, etc.) is contemplated herein.

In some aspects, a pneumatic stimulation system is provided, comprising a flow device, a patient interface fluidly coupled to the flow device via at least one conduit, and a controller operably connected to the flow device, and configured to receive sensor data from at least one sensor coupled to at least one of the user, the conduit, and the flow device, and automatically adjust a control setting based at least in part on the sensor data.

In some aspects, a method of pneumatic stimulation is provided, comprising providing a set of pulses of a gas to a user to pneumatically stimulate a hypoglossal nerve of the user, signaling the hypoglossal nerve to move the tongue forward, relieving the airway obstruction and allowing uninterrupted airflow. In some aspects, providing the set of pulses comprises providing the set of pulses via a nasal cannula. In some aspects, providing the set of pulses comprises providing the set of pulses via an oral mask. In some aspects, providing the set of pulses comprises providing the set of pulses via a flow generation system based on at least one control setting. In some aspects, the at least one control setting comprises at least one of a breath trigger setting, a boost flow setting (e.g., a boost flow rate setting), a boost flow duration setting, a base flow setting (e.g., a base flow rate setting), and an auto trigger timer.

In some aspects, a pneumatic stimulation system is provided, comprising a flow device, a patient interface fluidly coupled to the flow device via at least one conduit, and a controller operably connected to the flow device, and configured to receive sensor data from at least one sensor coupled to at least one of the user, the conduit, and the flow device, and determine at least one preferred control setting based at least in part on the sensor data. In some aspects, the preferred control setting can be suitable to prevent or treat sleep apnea and/or other sleep disordered breathing. In some aspects, the preferred control setting can be a preferred breath trigger setting (e.g., a setting between from 0.5 LPM to 5LPM), a preferred boost flow setting (e.g., a setting between 0 to 60 LPM), a preferred boost flow duration setting (e.g., a setting between between 0-1.5 seconds), a preferred base flow setting (e.g., a setting between 0-30 LPM), and a preferred auto trigger timer setting (e.g., a setting between 0.5 seconds to 30 seconds), or any other suitable flow/pressure/volume related setting.

In some aspects, some or all control settings can be selected randomly/randomized. In some aspects, artificial intelligence is utilized, and control settings can be adjusted based on trending data (e.g., automatically by the system, in real-time). The trending data can be, for example, detection or indication of hypopnea, degree of hypopnea, degree of apnea, effectiveness of control settings (e.g., in maintaining SpO₂), any other suitable trending data, and/or a combination thereof.

Other advantages and benefits of the disclosed assemblies, components and methods will be apparent to one of ordinary skill with a review of the following detailed description.

### BRIEF DESCRIPTION OF THE DRAWINGS

The details of embodiments of the present disclosure, both as to their structure and operation, may be gleaned in part by study of the accompanying drawings, in which like reference numerals refer to like parts, and in which:

FIG. 1 illustrates an exemplary sleep study setup;

FIG. 2 illustrates some exemplary control parameters and settings for certain systems and methods of the disclosure (but it should be appreciated that there are several additional control parameters and settings contemplated in addition to the ones shown in FIG. 2);

FIG. 3 illustrates one epoch from overnight sleep waveform without hypoglossal nerve stimulation (about 12 minutes and 39 seconds between 1:44:59 am and 1:57:38 am);

FIG. 4 illustrates one epoch from overnight sleep waveform with hypoglossal nerve stimulation using a system and method described herein (about 12 minutes and 37 seconds between 1:07:33 am and 1:20:11 am);

FIG. 5 illustrates a sample overnight sleep waveform I;

FIG. 6 illustrates a sample overnight sleep waveform II;

FIG. 7 illustrates an example of a patient interface;

FIG. 8 illustrates an epoch from overnight sleep waveform with hypoglossal nerve stimulation based on SpO₂ measurements and an algorithm to detect, reduce and/or prevent obstructive sleep apnea events;

FIG. 9 illustrates an epoch from overnight sleep waveform with hypoglossal nerve stimulation based on breath detection period and an algorithm to detect, reduce and/or prevent obstructive sleep apnea events;

FIG. 10 illustrates an epoch from overnight sleep waveform with hypoglossal nerve stimulation based on breath detection period using an algorithm to maintain and optimize patient comfort;

FIG. 11 illustrates a pneumatic stimulation system comprising a high flow therapy (HFT) device (or other flow device) with an integrated humidifier, tubing, and a patient interface, according to an embodiment;

FIG. 12 illustrates another pneumatic stimulation system comprising a HFT device (or other flow device), a standalone humidifier, tubing, and a patient interface, according to another embodiment;

FIG. 13 illustrates an example infrastructure, in which one or more of the processes described herein, may be implemented, according to an embodiment; and

FIG. 14 illustrates an example processing system, by which one or more of the processes described herein, may be executed, according to an embodiment.

### DETAILED DESCRIPTION

After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

In an aspect of the disclosure, a flow generation system is provided comprising a flow device and a patient interface. In some aspects, the flow generation system can comprise some or all components described in Applicant's U.S. 2016/0287824, U.S. Pat. Nos. 10,525,222, and 10,869,977. In some aspects, the flow generation system can comprise an HFT 150 heated high-flow system by Invent Medical Corporation.

Flow Devices

Some contemplated flow devices include a flow/pressure generator (a flow generation device and/or a pressure generation device). The following hardware combinations, among others, can be used in contemplated systems and methods under the algorithms described herein.

Combination 1. A blower as a flow/pressure generator, which optionally does not require a high-pressure source, sensors (e.g., one or more flow sensors, one or more pressure sensors), PCB, electronics, and/or other components.

Combination 2. A proportional valve as a flow/pressure generator, which requires a high-pressure source, sensors (e.g., one or more flow sensors, one or more pressure sensors), PCB, electronics, and/or other components.

Some contemplated flow devices include flow interrupters, which can have various configurations. In some aspects, the flow interrupters can comprise a stepper motor and slide valve, along with other components such as one or more flow sensors, pressure sensors, other sensors, electronics, PCB, and/or other components.

Other mechanisms are also contemplated for achieving the methods under the algorithms contemplated herein, including on-off solenoid valves with flow sensors, pressure sensors, and electronics. In some embodiments, a flow device can comprise a pressure source with different control mechanisms (e.g., valves).

Patient Interface

Various patient interfaces are contemplated for use in the disclosed systems and methods, including, for example, a mouthpiece, a mask (full face, nasal, pillow, oro-nasal, oral, total mask, combination thereof), or a hybrid interface (e.g., a cannula integrated into a pillow). The patient interface can be an individual patient interface such as a nasal cannula or mask, or a combination of an individual patient interface and heat moisture exchange (HME).

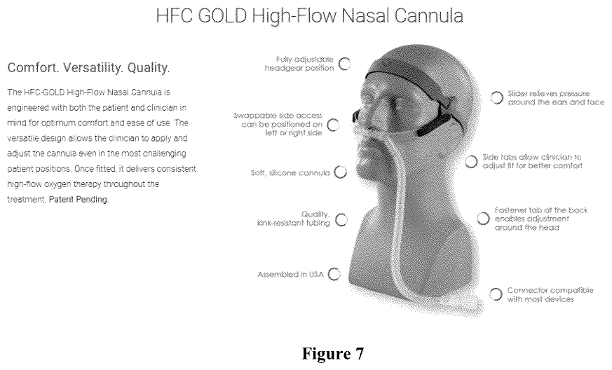

In some embodiments, a nasal cannula may be preferred as a patient interface as it is simpler and lighter, and more comfortable for a wearer. An example nasal cannula is shown in FIG. 7. While CPAP machines require a mask that provides a seal, the devices, systems and methods described herein can be used with a nasal cannula without a mask (e.g., as a greater amount of leakage is acceptable). CPAP is based on attempting to control pressure in the airway where leaks are problematic, while in some embodiments the systems disclosed herein can advantageously be independent of pressure (e.g., based on pulsed volume or flow/flow rate). Another major difference between CPAP machines and some contemplated devices and systems of the disclosure is that the CPAP machine's leaks vary throughout the breathing phases and are not constant/near constant. The devices, systems and methods described herein can also be used with, for example, an oral mask. While traditional treatments require that the patient keep their mouth closed (e.g., to prevent air from getting out and causing flow rate to increase and cause dry mouth and discomfort), the devices, systems and methods described herein can be used with a mouth open wide/without a chin strap.

It is contemplated that similar results would be achieved via other patient interfaces that are similar to a nasal cannula (e.g., a nasal pillow mask).

Control

In some aspects, the systems and methods described herein stimulate at least one of a hypoglossal nerve and one or more muscles that support the soft tissue in one's throat (e.g., tongue, soft palate). In some aspects, the systems and methods described herein can help prevent one's tongue or soft palate (or other structure) from obstructing their airway. Viewed from another perspective, the systems and methods described herein can comprise pulse flow rate/volume/pressure delivery of a gas. In some aspects, a high frequency component via flow rate and pressure can be embedded to the systems and methods described herein. The high frequency component can be construed as an AC component on top of DC (e.g., systems and methods described herein) component.

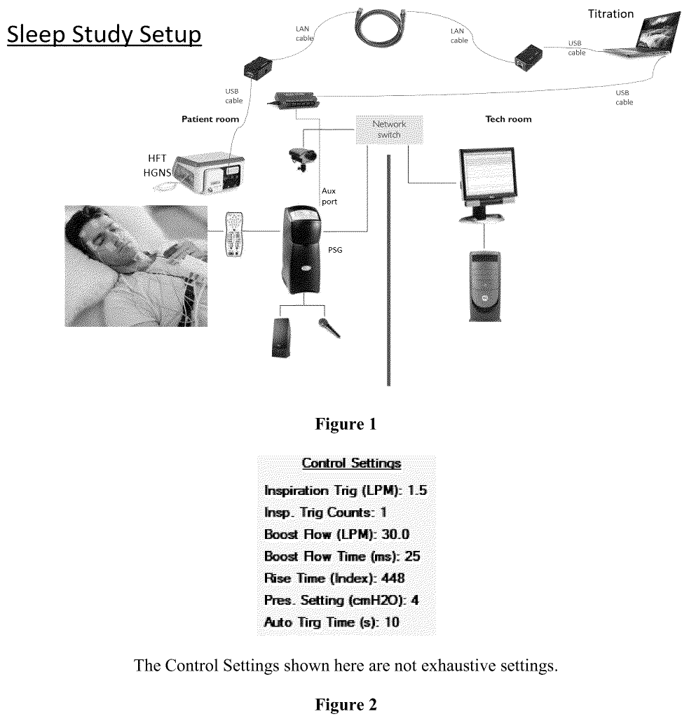

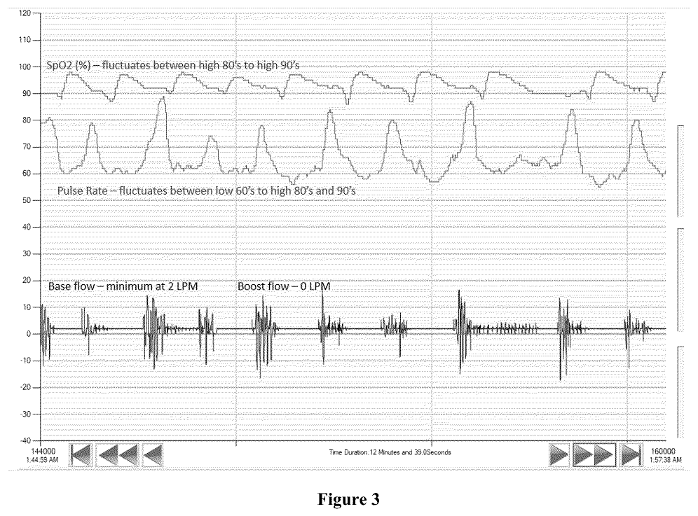

Applicant's pulsed gas flow rate/volume/pressure delivery algorithm has been tested on a selected subject and proven to prevent SpO2 drop/maintain SpO2 (read via a pulse oximeter) and to reduce apnea events significantly. FIG. 1 illustrates an example polysomnography (PSG) sleep study setup (as utilized for Applicant's sleep study described herein) with certain components in a “Patient room” communicatively coupled to components in a “Tech room”. Titration (e.g., determining the correct device settings for a person) can happen in a room separate from the sleep subject. The PSG system can record various activity/stats, for example, brain waves, oxygen level in blood, heart rate, breathing, eye movement, and/or leg movement. Control settings were set up (example shown in FIG. 2) in the Tech room, and results from the sleep study can be recorded in the Tech room. In the study described here, settings were adjusted for multiple nights to determine optimal treatment.

There can be several key settings, which can be adjusted. For example, the setting for a breath trigger control parameter (Inspiration Trig) can be set from 0.5 LPM to 5LPM, a constant trigger, or an adaptive/auto/self-adjusting trigger. The boost flow parameter can be set from 0 to 60 LPM, a constant flow rate, or an adaptive/auto/self-adjusting flow rate. The boost flow duration parameter (boost flow time) can be set, for example, between 0-1.5 seconds, and can be a constant duration or an adaptive/auto, self-adjusting duration. The base flow parameter can be from 0-30 LPM, and can be a constant flow rate or an adaptive/auto/self-adjusting flow rate. The auto-trigger timer parameter (Auto Trig Time) can be set from 0.5 seconds to 30 seconds, and can be a constant timer or an adaptive/auto/self-adjusting timer. The auto trigger flow parameter can comprise a constant flow or an adaptive/auto/self-adjusting flow.

In some contemplated systems, a ramp feature can be provided to make the initial phase of therapy more comfortable. In some contemplated systems, a ramp feature can be provided for a decrease in one or more settings. In some embodiments, the ramp time parameter (the period during which pressure increases to the full therapy setting) can be set, for example, between 0 and 45 minutes. A ramp feature can delay the full therapy settings and allow for a gradual increase, which can be, for example, linear, exponential, or stepwise. The initial setting can be a base flow with zero boost flow and/or pressure.

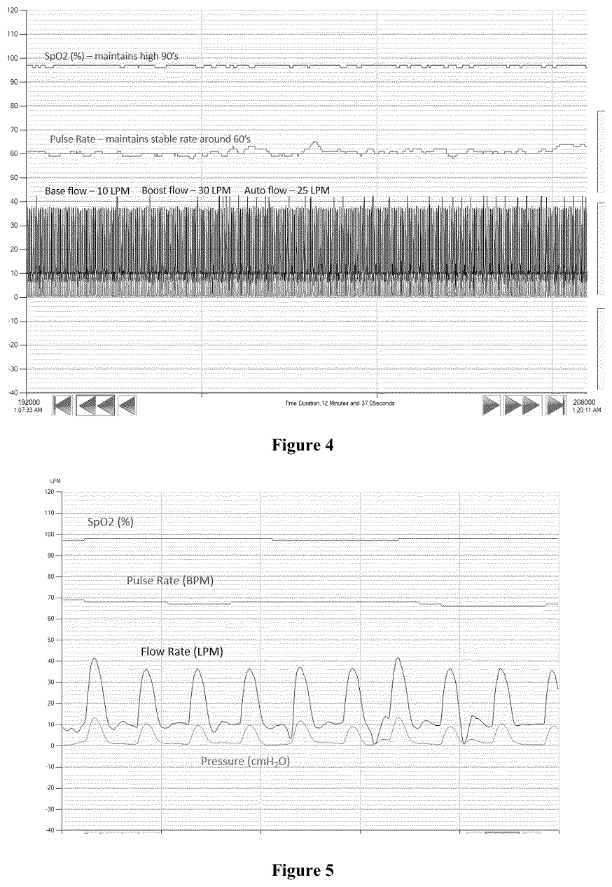

FIG. 3 shows one epoch from overnight sleep waveform without hypoglossal nerve stimulation (about 12 minutes and 39 seconds between 1:44:59 am and 1:57:38 am). The epochs illustrated herein are based on the pulse oximeter readings (SpO₂ and pulse rate), flow rate and pressure recordings. It should be appreciated that typical sensors used in sleep studies can include, for example, electrocardiograms (EKG), electro-encephalogram (EEG), electrooculography (EOG), electromyography (EMG), piezoelectric sensors, acoustic sensors, body position sensor, cameras, microphones, which are optional in systems and methods described herein, as well as any other suitable sensors. The top line reflects SpO2(%) fluctuating between high 80 s and high 90 s. The second line from the top reflects pulse rate fluctuating between low 60 s to high 80 s and 90 s. The third line from the bottom reflects a flow rate. In this case, there is no boost flow. The bottom line reflects pressure. The base flow, boost flow, and auto flow can all be set/adjusted. The base flow is a constant flow of some gas from the flow device. The boost flow is triggered by patient effort, and the auto flow is triggered by time. Sudden drops in blood oxygen levels during sleep apnea increase blood pressure and strain the cardiovascular system. Obstructive sleep apnea increases the risk of high blood pressure (hypertension). FIG. 4 shows one epoch from overnight sleep waveform with hypoglossal nerve stimulation using the systems and methods disclosed herein (about 12 minutes and 37 seconds between 1:07:33 am and 1:20:11 am). The overnight treatment depicts a significant reduction in apnea events and much-improved SpO2 during the entire sleep duration. SpO2(%) maintained in the high 90 s, and pulse rate maintains stable rate around 60 s. Base flow (a continuous flow) was set to 10 LPM. Boost flow (pulsed flow) was set to 30 LPM. In some embodiments, it is contemplated that boost flow can be provided without a base flow. Auto flow was set to 25 LPM. The control settings shown in FIG. 4 are an illustrative set of settings provided as an example, and do not necessarily represent the optimum settings. The optimum settings will vary, for example, from patient to patient, from night to night. The hyoglossus muscle depresses and retracts the tongue and is innervated by the hypoglossal nerve. Pneumatic stimulation of the hypoglossal nerve as described herein can advantageously dilate the pharyngeal airway and prevent and/or reduce airway obstruction. A reduction in apnea hypopnea index (AHI) and a reduction in apneic events via contemplated systems and methods was shown via a sleep study as shown in FIGS. 3-4. When obstructive sleep apnea occurs, the SpO2 drops from the SpO2's baseline, and the pulse rate accelerates from the pulse rate's baseline with a phase shift. Some known sleep apnea devices claim to reduce AHI. The AHI is a diagnostic tool for determining the presence and severity of obstructive sleep apnea. There is generally agreement on the definition of an apnea. Hypopnea is more subjective and there is no standard measurement for what counts as a hypopnea. Thus, different definitions of hypopnea can lead to different AHI scores. Improvement in the hypopnea index may not represent an improvement in SpO₂, but apnea improvement/steady SpO₂ (at non-apnea levels) has clinical benefits.

Algorithms for pulsed volume delivery of a gas include (a) a flow-based method —amplitude, duration, base flow, which was used for the sleep study described herein.

Flow-based method. As noted above, there can be several key parameters, for which the settings can be adjusted. For example, the breath trigger parameter (Inspiration Trig) can be set between 0.5 LPM to 5LPM (or any other suitable flow rate), and can be a constant trigger, or an adaptive/auto/self-adjusting trigger. The boost flow parameter can be set between 0 to 60 LPM (or any other suitable flow rate), and can be a constant flow, or an adaptive/auto/self-adjusting flow. The boost flow duration parameter (boost flow time) can be set, for example, between 0.1-1.5 seconds (or any other suitable duration), and can be a constant duration or an adaptive/auto, self-adjusting duration. The base flow parameter can be from 0-30 LPM (or any other suitable flow rate), and can be a constant flow or an adaptive/auto/self-adjusting flow. The auto-trigger timer parameter (Auto Trig Time) can be set from 0.5 seconds to 30 seconds (or any other suitable interval), and can be a constant timer or an adaptive/auto/self-adjusting timer. The auto trigger flow parameter can comprise a constant flow or an adaptive/auto/self-adjusting flow.

In some contemplated systems, a ramp feature can be provided to make the initial phase (or wind down phase) of therapy more comfortable. In some embodiments, the ramp time (the period during which pressure increases to the full therapy setting) can be set, for example, between 0 and 45 minutes (or any other suitable amount of time). A ramp feature can delay the full therapy settings and allow for a gradual increase and/or decrease. The initial setting can be a base flow with zero boost flow and/or pressure.

Pulse volume delivery of a gas, either via boost flow or boost pressure can be a single pulse delivery or multiple pulse delivery. Some exemplary types of pulses can be square, ascending ramp, descending ramp, sinusoidal, or any other suitable waveshape.

Control Parameters.

There are numerous potential control parameters, including those shown in FIG. 2 (Inspiration Trig, Inspiration Trig Counts, Boost Flow, Boost Flow Time, Rise Time, Pressure Settings, Auto Trig Time) and those not shown in FIG. 2 (for example, apnea boost flow, apnea range, ramp time,- sleep state (e.g., fallen asleep, awoken), which can be manually adjusted or auto-adjusted, for example, based on trends of the patient, other individuals, and/or machine learning.

In some embodiments, control parameters can primarily be grouped into three main parameters: flow amplitude; flow duration; and flow frequency. Each of the three primary parameters can be grouped into additional groups, for example, titration setting, random setting, and auto setting, as follows:
Flow Amplitude
   Flow amplitude titration setting
   Flow amplitude random setting/generation
   Flow amplitude auto setting/control (adjusted based on how the patient is breathing).
Flow Duration
   Flow duration titration setting
   Flow duration random setting/generation
   Flow duration auto setting/control
Flow Frequency
   Flow frequency titration setting
   Flow frequency random setting/generation
   Flow frequency auto setting/control

It is contemplated that flow can be replaced with a pressure or volume. Pressure and volume can be the “dual” of flow. (Gas) volume over time is flow rate. (Gas) volume over a defined space is pressure. As such, flow can be derived from volume or pressure. Volume or pressure can yield flow via proper manipulation or control. Viewed from another perspective, it is contemplated that either pressure or volume can take the exact same form of delivery as flow.

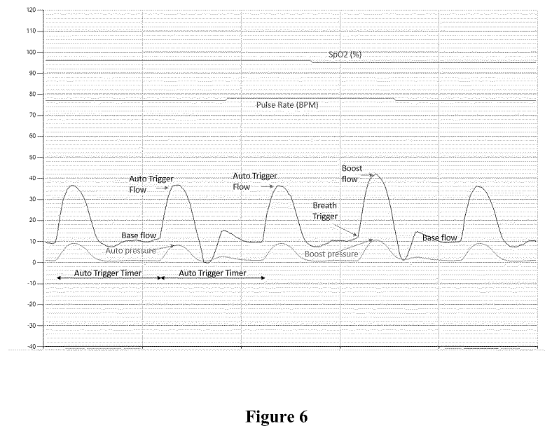

Sample overnight sleep waveform I, is shown in FIG. 5, and sample overnight sleep waveform II, is shown in FIG. 6. A pulse delivery can be viewed as a non-continuous delivery. As illustrated in FIG. 6, an auto trigger timer can be implemented, which can cause a machine pulse delivery based on a time (e.g., every second, every two seconds, every three seconds, every four seconds, every five seconds, every 0.5-5 seconds, every 0.5-4 seconds, every 0.5-3.5 seconds, every 0.5-2.5 seconds, every 0.5-1.5 seconds, every 1.5-3.5 seconds, every 1.5-2.5 seconds). As illustrated in FIG. 6, systems and methods of the disclosure can also comprise pulse delivery based on patient effort (as shown with the boost flow and boost pressure based on a breath trigger, which can be set with the control settings).

Other contemplated algorithms for pulse delivery include, as examples, (b) pressure-based method—dual of flow/volume—amplitude, duration, (c) preemptive and/or auto adjustment, for example, based on hypopnea and/or apnea, and (d) synchronizing with patient inhalation effort. (e) at random—random flow/volume/pressure—amplitude, duration, frequency, trigger sensitivity.

Pressure-based Method

It should be appreciated that in contemplated systems and methods, pressure and/or flow can be an input. With the pressure-based method, similarly to the flow-based method, several key settings for various parameters can be set and/or adjusted.

The base pressure—dual of base flow—can be for example from 0-10 centimeters of water (cmH₂O). The base pressure can be a constant pressure, or an adaptive/auto/self-adjusting pressure.

The boost pressure—dual of boost flow—can be for example from 0 to 30 cmH₂O. The boost pressure can be a constant pressure, or an adaptive/auto/self-adjusting pressure.

The boost pressure duration—dual of boost flow duration—can be for example from 0-1.5 seconds. The boost pressure duration can be a constant duration, or can be an adaptive/auto/self-adjusting duration.

The breath trigger can be, for example, from 0.5 LPM to 5 LPM, and a constant trigger, or adaptive/auto/self-adjusting trigger, or from 0.2 cmH₂O to 3 cmH₂O, and a constant trigger, or an adaptive/auto/self-adjusting trigger.

The auto-trigger timer can be, for example, from 0.5 seconds to 30 seconds, and can be a constant timer, or an adaptive/auto/self-adjusting timer.

The auto-trigger pressure—dual of auto-trigger flow—can be a constant pressure, or an adaptive/auto/self-adjusting pressure.

The pulse volume delivery, via a boost flow or boost pressure, can be a single pulse delivery or multiple pulse delivery. The types of pulses can be square, ascending ramp, descending ramp, sinusoidal, or any other suitable waveshape.

Pre-emptive and/or Auto Adjustment

The devices, systems and methods described herein can provide volume pulse after a certain time elapses without normal breathing (e.g., apnea, hypopnea). In some contemplated aspects, preemptive pulse delivery based on hypopnea detection (decreasing flow rate) is contemplated (before apnea event, preemptively deliver volume pulse). In some aspects, an automatic adjustment can be made based on a reduction in flow, e.g., a 30%, 50%, 100% (or any other suitable) increase, or a decrease based on a recovery.

In some aspects, upon detection of an event (e.g., hypopnea, apnea), any or all settings can be adjusted (e.g., automatically). For example, upon detection of a hypopnea event, any or all settings can be adjusted to alleviate or reduce the hypopnea and/or prevent apnea. Hypopnea detection based on a reduction in flow rate can be a challenge where the systems and methods described herein control the flow rate. In some aspects, hypopnea detection can be based on the motor/blower speed. Trending the motor/blower speed data could indicate non-hypopnea breaths. When the motor/blower speed increases due to the obstruction of the airway, i.e., increased resistance, the presence of hypopnea can be deduced or detected. In some aspects, hypopnea detection can be based on a flow rate, e.g., a reduction in flow rate. Trending the reduction of flow rate data could indicate the state of upper airway obstruction. If the motor/blower speed indicates the degree of reduction in flow rate, the devices, systems and methods herein can adjust several settings (e.g., set/control flow rate, frequency of pulse delivery, or randomness of the pulse delivery.

In some aspects, artificial intelligence (e.g., machine learning) algorithms are provided and the systems and methods herein can be used to learn sleep patterns in real-time and continually adjust the setting parameters.

In an embodiment, systems, methods, and non-transitory computer-readable media are disclosed for pneumatic stimulation to prevent sleep apnea and/or apneic events and/or other sleep disordered breathing events.

Algorithm to Detect, Reduce and/or Prevent Obstructive Sleep Apnea Events.

An objective of the disclosure is to detect, reduce and/or prevent obstructive sleep apnea events, thereby reducing or minimizing the incidence and/or incidents of SpO₂ falling below the baseline (non-obstructive state) during sleep.

Apnea detection and a delivery of a pulse, a set of pulses, and/or sets of pulses of gas can be based on, for example, SpO₂ measurements, breath detection periods, or any other suitable mechanisms.

For example, apnea can be predicted and/or detected when the SpO₂ measurement drops below the baseline SpO₂ by a predetermined percentage, for example, by ≥1%, ≥2%, ≥3%, ≥5%, or ≥8%. This drop threshold can be settable or auto-adjustable/detectable. For example, if apnea detection is set for 10%, based on a user's SpO₂ and/or other measurements over time, apnea detection settings can be auto-adjusted to be set for 5%. In some aspects, a single drop in SpO₂ (and/or apnea event) can be used to adjust control parameter settings. In some aspects, multiple drops in SpO₂ (and/or multiple apnea events) can be used to control parameter settings. In some aspects, use of either a single or multiple drops in SpO₂ (and/or apnea events) can be used to make adjustments of the control parameter via a settable or auto-adjustable/detectable algorithm.

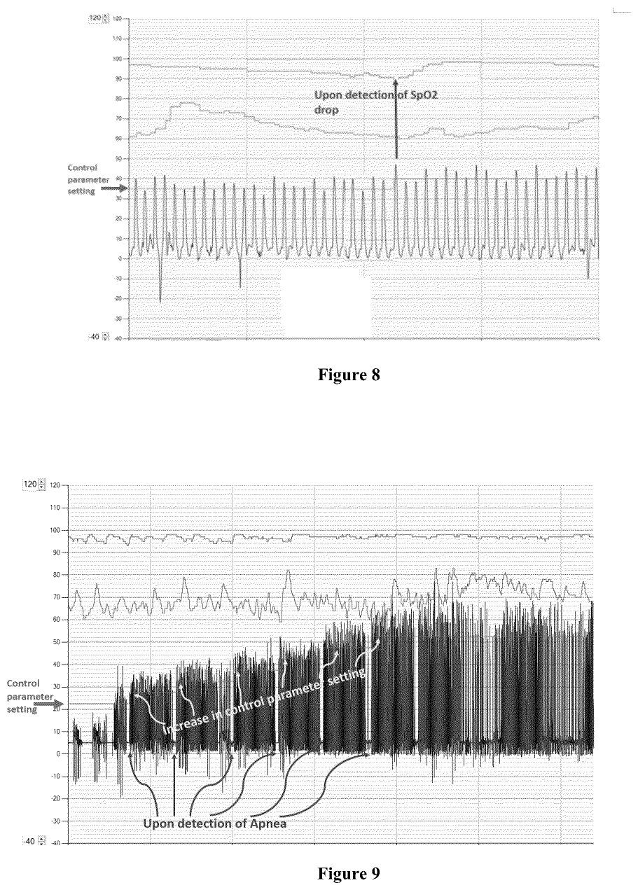

FIG. 8 illustrates an epoch from overnight sleep waveform with hypoglossal nerve stimulation based on SpO₂ measurements and an algorithm to detect, reduce and/or prevent obstructive sleep apnea events. The top line reflects SpO₂ (%) fluctuating between high 80 s/low 90 s and high 90 s. The second line from the top reflects pulse rate fluctuating between low 60 s to high 70 s. The third line from the top (between about 35-40) represents “boost flow” as a control parameter setting. The bottom line (pulsed line) reflects a main flow of pulses of gas (e.g., pressurized air). This is Boost Flow around the Boost Flow control setting with±Boost Flow Range (in this illustration, it is set at 5 LPM) random variation. As illustrated in FIG. 8, upon a detection of an SpO₂ drop beyond a threshold amount from a baseline, the “boost flow” control parameter setting is increased (here, from about 35 to about 40), which increases the boost flow of the pulses. What is shown is random flow around the boost flow setting, which is 35 LPM before the increase. After the increase, upon the detection of the reduction of the SpO₂, the boost flow is increased to 40 LPM.

As another example, apnea can be predicted and/or detected based on one or more breath detection periods (with or without SpO₂ data), for example, when patient trigger efforts are not detected for a predetermined period of time (e.g., 10 seconds), which can be settable or auto-adjustable/detectable. Adjustments based on breath detection can be advantageous as patients typically do not wear pulse oximeters at home, at least not consistently. For example, if apnea prediction and/or detection is set for instances of 10 or more continuous seconds where patient trigger efforts are not detected, based on a user's pattern of trigger efforts and/or other measurements over time, apnea prediction and/or detection settings can be auto-adjusted to be set for, for example, ≥5 seconds. In some aspects, a single segment of time can be used to adjust control parameter settings. In some aspects, use of multiple segments of time can be used to control parameter settings. In some aspects, use of either a single or multiple segments of time can be used to make adjustments of the control parameter via a settable or auto-adjustable/detectable algorithm.

FIG. 9 illustrates an epoch from overnight sleep waveform with hypoglossal nerve stimulation based on breath detection periods and an algorithm to detect, reduce and/or prevent obstructive sleep apnea events. The top line reflects SpO2(%) fluctuating between low/mid 90 s and high 90 s. The second line from the top reflects pulse rate fluctuating between high 50 s to low 80 s. The third line from the top (between about 22-52) represents “boost flow” as a control parameter setting. The bottom line reflects a main flow of pulses of gas (e.g., pressurized air). As illustrated in FIG. 9, upon a detection of a lack of patient trigger efforts beyond a threshold amount (e.g., 5 seconds), the “boost flow” control parameter setting is increased (here, multiple times, from about 22 to about 28, from about 28 to about 32, from about 32 to about 38, from about 38 to about 42, from about 42 to about 48, and from about 48 to about 52), which increases the Boost Flow control setting of the pulses.

Here the control parameter setting is gradually increased (multiple increases in control parameter setting). In some embodiments, based on historical patterns/trends (e.g., having to increase boost flow settings to a maximum level) or based on a single or multiple apnea events, the system can be configured to auto adjust, for example, to increase the boost flow setting by a greater amount more quickly.

Algorithm to Maintain and Optimize Patient Comfort.

Another object of the disclosure is to make the person comfortable during sleep (which may also be referred to as a treatment period).

Delivery of a pulse, a set of pulses, and/or sets of pulses of gas to maintain and optimize patient comfort can be based on, for example, SpO₂ measurements, breath detection periods, or any other suitable mechanisms.

Similarly to the algorithms for detecting, reducing and/or preventing obstructive sleep apnea, algorithms for maintaining and optimizing patient comfort can cause the system to deliver a pulse, a set of pulses, and/or sets of pulses of gas based on, for example, SpO₂ measurements (e.g., reduction of a Boost Flow control setting based on not detecting a drop in SpO₂ over a single or multiple segments of time), breath detection periods, or any other suitable mechanisms.

In some aspects, a single segment of time can be used to adjust control parameter settings. In some aspects, use of multiple segments of time can be used to control parameter settings. In some aspects, use of either a single or multiple segments of time can be used to make adjustments of the control parameter via a settable or auto-adjustable/detectable algorithm.

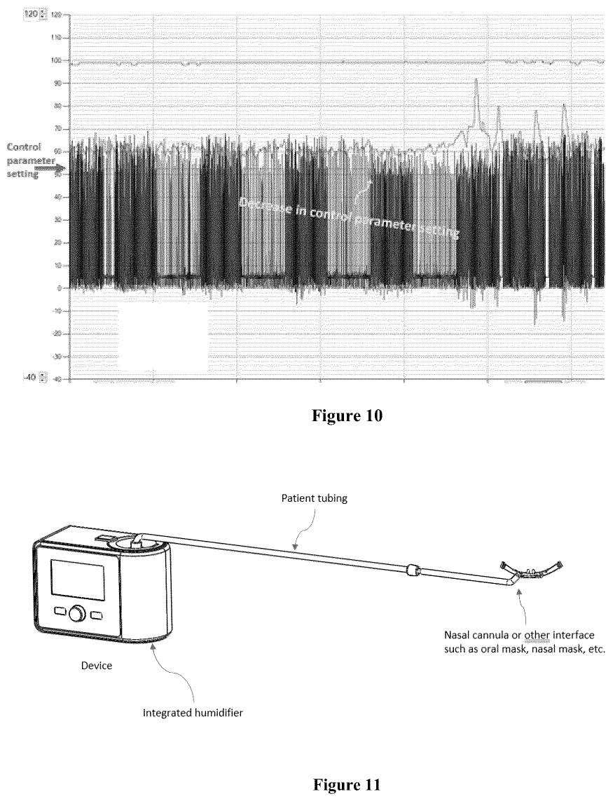

FIG. 10 illustrates an epoch from overnight sleep waveform with hypoglossal nerve stimulation based on breath detection period using an algorithm to maintain and optimize patient comfort. The top line reflects SpO2(%) being stable in the high 90 s. The second line from the top reflects pulse rate fluctuating between high 50 s to low 90 s. The third line from the top (starting at about 52.5, decreasing to about 50 and increasing to about 55) represents a control parameter setting (here, boost flow). The bottom line reflects a main flow of pulses of gas (e.g., pressurized air), which here is Boost Flow plus Base Flow. As illustrated, a control parameter setting is adjusted (decreased for greater comfort—e.g., when no apnea events are detected, then boost flow is decreased) based on the pulse rate of the patient. In the example shown, the boost flow is decreased by 50% of the increase. The increase shown is by 5 LPM, and the decrease shown is by 2.5 LPM. However, it should be appreciated that the decrease(s) and/or increase(s) can be of any suitable amount(s). For example, a decrease can comprise between 5-75% of an increase amount.

While the examples above are primarily directed to detection, prevention and/or treatment or obstructive sleep apnea, it should be appreciated that the devices, systems and methods described herein can be used to detect, prevent, and/or treat other sleep disordered breathing events.

Pneumatic Stimulation Systems.

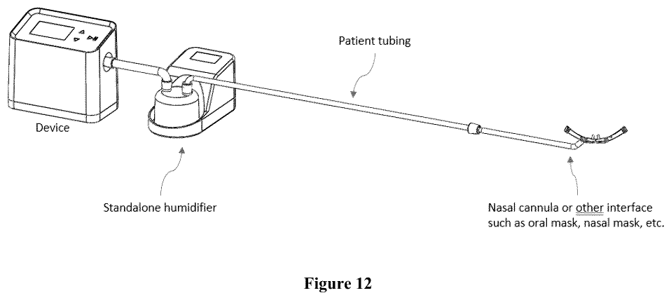

FIGS. 11-12 illustrate two different exemplary embodiments of a pneumatic stimulation system. The system of FIG. 11 includes a flow device (e.g., a HFT device) with an integrated humidifier, tubing, and a patient interface. The system of FIG. 12 has a humidifier separate from the flow device. In some embodiments, the system comprises one or more sensors. In some embodiments, the flow device comprises and/or is coupled to a one or more processors configured to receive sensor data from one or more sensors (e.g., record sleep activity). The one or more processors can be configured to obtain data associated with sleep patterns of the patient and/or other individuals. The one or more processors can be configured to predict or detect an apnea, hypopnea and/or other event based on at least one of the sensor data from the one or more sensors worn by a patient and data associated with sleep patterns of the patient and/or other individuals. In some embodiments, the flow device comprises and/or is coupled to a controller, and the one or more processors can be configured to cause the controller to adjust one or more settings based on the sensor data or any other suitable data (e.g., from the patient, from other individuals, trend data, historical data, and/or any other suitable data).

System Overview

### 1. Infrastructure

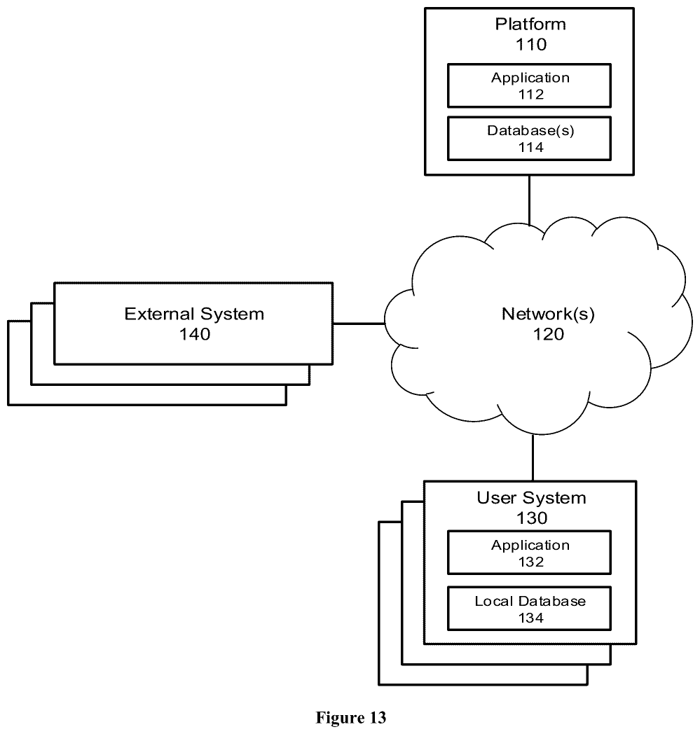

FIG. 13 illustrates an example infrastructure in which one or more of the disclosed processes may be implemented, according to an embodiment. The infrastructure may comprise a platform 110 (e.g., one or more servers) which hosts and/or executes one or more of the various functions, processes, methods, and/or software modules described herein. Platform 110 may comprise dedicated servers, or may instead comprise cloud instances, which utilize shared resources of one or more servers. These servers or cloud instances may be collocated and/or geographically distributed. Platform 110 may also comprise or be communicatively connected to a server application 112 and/or one or more databases 114. In addition, platform 110 may be communicatively connected to one or more user systems 130 via one or more networks 120, or may be entirely implemented on the loopback (e.g., localhost) interface. Platform 110 may also be communicatively connected to one or more external systems 140 (e.g., other platforms, websites, etc.) via one or more networks 120.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases. In addition, communication between any of these systems, for example, platform 110, user systems 130, and/or external system 140, may be entirely implemented on the loopback (e.g., localhost) interface.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, and/or the like. Each user system 130 may comprise or be communicatively connected to a client application 132 and/or one or more local databases 134. While user system 130 and platform 110 are shown here as separate devices connected by a network 120. User system 130 may comprise an application 132 that may comprise one portion of a distributed cloud-based system that integrates with platform 110, for example, using a multi-tasking OS (e.g., Linux) and local only (localhost) network addresses.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, or may be entirely implemented on the loopback (e.g., localhost) interface, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. Platform 110 may also respond to other requests from user system(s) 130.

Platform 110 may comprise, be communicatively coupled with, or otherwise have access to one or more database(s) 114. For example, platform 110 may comprise one or more database servers which manage one or more databases 114. Server application 112 executing on platform 110 and/or client application 132 executing on user system 130 may submit data (e.g., user data, form data, etc.) to be stored in database(s) 114, and/or request access to data stored in database(s) 114. Any suitable database may be utilized, including without limitation MySQL™, Oracle™ IBM™, Microsoft SQL™, Access™, PostgreSQL™, MongoDB™, and the like, including cloud-based databases and proprietary databases. Data may be sent to platform 110, for instance, using the well-known POST, GET, and PUT request supported by HTTP, via FTP, proprietary protocols, requests using data encryption via SSL (HTTPS requests), and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from external system(s) 140, and provide responses in eXtensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes, methods, functionality, storage, and/or the like, described herein. For example, in such an embodiment, a client application 132, executing on one or more user system(s) 130, may interact with a server application 112 executing on platform 110 to execute one or more or a portion of one or more of the various functions, processes, methods, and/or software modules described herein. In an embodiment, client application 132 may utilize a local database 134 for storing data locally on user system 130.

Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the software described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules comprising instructions that implement one or more of the processes, methods, or functions described herein.

While platform 110, user systems 130, and external systems 140 are shown as separate devices communicatively coupled by network 120, each of the devices shown as platform 110, user systems 130, and external systems 140 may be implemented on one or more devices, and/or one or more of platform 110, user systems 130, and external systems 140 may be implemented on a single device.

### 2. Example Processing Device

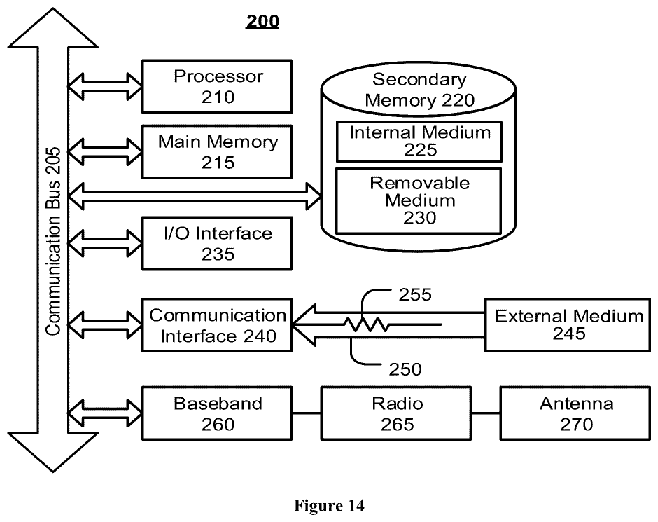

FIG. 14 is a block diagram illustrating an example wired or wireless system 200 that may be used in connection with various embodiments described herein. For example, system 200 may be used as or in conjunction with one or more of the functions, processes, or methods (e.g., to store and/or execute the software) described herein, and may represent components of platform 110, user system(s) 130, external system(s) 140, and/or other processing devices described herein. System 200 can be a server or any conventional personal computer, or any other processor-enabled device that is capable of wired or wireless data communication. Other computer systems and/or architectures may be also used, as will be clear to those skilled in the art.

System 200 preferably includes one or more processors 210. Processor(s) 210 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 210. Examples of processors which may be used with system 200 include, without limitation, any of the processors (e.g., Pentium™, Core i7™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor 210 is preferably connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/5-100, and/or the like.

System 200 preferably includes a main memory 215 and may also include a secondary memory 220. Main memory 215 provides storage of instructions and data for programs executing on processor 210, such as any of the software discussed herein. It should be understood that programs stored in the memory and executed by processor 210 may be written and/or compiled according to any suitable language, including without limitation C/C++, Java, JavaScript, Perl, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 220 is a non-transitory computer-readable medium having computer-executable code (e.g., any of the software disclosed herein) and/or other data stored thereon. The computer software or data stored on secondary memory 220 is read into main memory 215 for execution by processor 210. Secondary memory 220 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 220 may optionally include an internal medium 225 and/or a removable medium 230. Removable medium 230 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

In alternative embodiments, secondary memory 220 may include other similar means for allowing computer programs or other data or instructions to be loaded into system 200. Such means may include, for example, a communication interface 240, which allows software and data to be transferred from external storage medium 245 to system 200. Examples of external storage medium 245 include an external hard disk drive, an external optical drive, an external magneto-optical drive, and/or the like.

As mentioned above, system 200 may include a communication interface 240. Communication interface 240 allows software and data to be transferred between system 200 and external devices (e.g. printers), networks, or other information sources. For example, computer software or executable code may be transferred to system 200 from a network server (e.g., platform 110) via communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 240 are generally in the form of electrical communication signals 255. These signals 255 may be provided to communication interface 240 via a communication channel 250. In an embodiment, communication channel 250 may be a wired or wireless network (e.g., network(s) 120), or any variety of other communication links. Communication channel 250 carries signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed software) is stored in main memory 215 and/or secondary memory 220. Computer-executable code can also be received via communication interface 240 and stored in main memory 215 and/or secondary memory 220. Such computer programs, when executed, enable system 200 to perform the various functions of the disclosed embodiments as described elsewhere herein.

In this description, the term “computer-readable medium” is used to refer to any non-transitory computer-readable storage media used to provide computer-executable code and/or other data to or within system 200. Examples of such media include main memory 215, secondary memory 220 (including internal memory 225, removable medium 230, and external storage medium 245), and any peripheral device communicatively coupled with communication interface 240 (including a network information server or other network device). These non-transitory computer-readable media are means for providing software and/or other data to system 200.

In an embodiment that is implemented using software, the software may be stored on a computer-readable medium and loaded into system 200 by way of removable medium 230, I/O interface 235, or communication interface 240. In such an embodiment, the software is loaded into system 200 in the form of electrical communication signals 255. The software, when executed by processor 210, preferably causes processor 210 to perform one or more of the processes and functions described elsewhere herein.

In an embodiment, I/O interface 235 provides an interface between one or more components of system 200 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet, or other mobile device).

System 200 may also include optional wireless communication components that facilitate wireless communication over a voice network and/or a data network (e.g., in the case of user system 130). The wireless communication components comprise an antenna system 270, a radio system 265, and a baseband system 260. In system 200, radio frequency (RF) signals are transmitted and received over the air by antenna system 270 under the management of radio system 265.

In an embodiment, antenna system 270 may comprise one or more antennae and one or more multiplexors (not shown) that perform a switching function to provide antenna system 270 with transmit and receive signal paths. In the receive path, received RF signals can be coupled from a multiplexor to a low noise amplifier (not shown) that amplifies the received RF signal and sends the amplified signal to radio system 265.

In an alternative embodiment, radio system 265 may comprise one or more radios that are configured to communicate over various frequencies. In an embodiment, radio system 265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 265 to baseband system 260.

If the received signal contains audio information, then baseband system 260 decodes the signal and converts it to an analog signal. Then the signal is amplified and sent to a speaker. Baseband system 260 also receives analog audio signals from a microphone. These analog audio signals are converted to digital signals and encoded by baseband system 260. Baseband system 260 also encodes the digital signals for transmission and generates a baseband transmit audio signal that is routed to the modulator portion of radio system 265. The modulator mixes the baseband transmit audio signal with an RF carrier signal, generating an RF transmit signal that is routed to antenna system 270 and may pass through a power amplifier (not shown). The power amplifier amplifies the RF transmit signal and routes it to antenna system 270, where the signal is switched to the antenna port for transmission.

Baseband system 260 is also communicatively coupled with processor(s) 210. Processor(s) 210 may have access to data storage areas 215 and 220. Processor(s) 210 are preferably configured to execute instructions (i.e., computer programs, such as the disclosed software) that can be stored in main memory 215 or secondary memory 220. Computer programs can also be received from baseband processor 260 and stored in main memory 210 or in secondary memory 220, or executed upon receipt. Such computer programs, when executed, can enable system 200 to perform the various functions of the disclosed embodiments.

### 3. Process Overview

Embodiments of processes for pneumatic stimulation to prevent sleep apnea and/or apneic events and/or other sleep disordered breathing events will now be described in detail. It should be understood that the described processes may be embodied in one or more software modules that are executed by one or more hardware processors (e.g., processor 210), for example, as a software application (e.g., server application 112, client application 132, and/or a distributed application comprising both server application 112 and client application 132), which may be executed wholly by processor(s) of platform 110, wholly by processor(s) of user system(s) 130, or may be distributed across platform 110 and user system(s) 130, such that some portions or modules of the software application are executed by platform 110 and other portions or modules of the software application are executed by user system(s) 130. The described processes may be implemented as instructions represented in source code, object code, and/or machine code. These instructions may be executed directly by hardware processor(s) 210, or alternatively, may be executed by a virtual machine operating between the object code and hardware processor(s) 210. In addition, the disclosed software may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for ease of description. Specific functions or steps can be moved from one component, block, module, circuit, or step to another without departing from the invention.

Furthermore, while the processes, described herein, are illustrated with a certain arrangement and ordering of subprocesses, each process may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

In order to detect or predict a hypopnea (or other) event and preemptively deliver a pulse and/or auto-adjust one or more settings, sensor data from one or more sensors of a system (e.g., one or more flow sensors, one or more pressure sensors, electrocardiogram, electro-encephalogram, microphone, sensor to measure snore volume, sensor to monitor body position) can be obtained by a user system 130. The user system can comprise a system 200 (e.g., PSG in FIG. 1) with a sensor interfaced, e.g., via I/O interface 235. The user system 130 can, when a hypopnea (or other) event is detected, communicate such detection, via communication interface 240 and/or radio 265. It is contemplated that an event can be detected in any suitable matter, for example, via a drop or rise in a measurement below or above a predetermined threshold. In some aspects, the predetermined threshold can automatically be updated based on machine learning algorithms and data obtained from one or more user systems and external systems. The detection can be, for example, communicated back to a platform 110 (e.g., Tech room of FIG. 1) operated by, or on behalf of a medical or sleep study facility. The platform can alert a user (e.g., a medical professional) so that they can adjust a setting. Additionally or alternatively, the platform can automatically cause an adjustment of a setting (e.g., one or more settings shown in FIG. 2) based on, for example, the detection, the sensor data from the one or more sensors of the system, and/or sensor data from one or more external systems.

In some aspects, the control settings can be fixed settings. During sleep lab titration, a sleep tech or other person can adjust control settings periodically to determine the optimum settings (e.g., with the use of sensor data). Once optimum settings are determined, the optimum settings for a specific patient can be prescribed. The optimum control settings determined may not be the optimum control settings for every night and may need to be adjusted.

In some aspects, the control settings can be Al based settings. Based on the trended information such as hypopnea or degree of hypopnea or apnea, the control settings can be adjusted automatically. For example, the pulse flow/volume delivery can be increased or decreased, the pulse delivery frequency can be increased or decreased or at random, the breath triggering sensitivity can be increased or decreased.

High-Frequency Method

Also contemplated herein are high-frequency mechanisms and algorithms. Hypoglossal nerve stimulation can also be achieved via a high-frequency flow or pressure delivery. The frequency can range, for example, from 1 Hz to 1 kHz. High-frequency can involve continuous stimulation of the hypoglossal nerve. High-frequency treatment can be composed with pulse delivery, in some contemplated embodiments herein. Exemplary high-frequency generating mechanisms include, among others, voice coil valves, stepper motors, loud speakers, or flow interrupting valves.

Synchronizing with Patient Inhalation Effort

In some aspects, the timing of boost flow or boost pressure delivery can be random. In some aspects, the timing of boost flow or boost pressure delivery can be synchronized with a patient's inhalation effort, exhalation effort, or any time in between. In some aspects, the timing of boost flow or boost pressure delivery can be set independent of the patient's inhalation and/or exhalation effort. In some aspects, the timing of boost flow or pressure flow delivery can be pre-programmed and/or self-adjusted/auto-adjusted. In some aspects, the timing of boost flow or boost pressure delivery can be synchronized with the patient's apnea detection or various derivatives of the patient's apnea detection.

Hybrid Methods

Hybrid high frequency+pulse mechanisms and algorithms are contemplated herein. In some aspects, the high-frequency component is an AC component, and flow boost or pressure boost is a DC component.

Other contemplated hybrid methods and algorithms include those that provide pulse volume delivery+high frequency, pulse volume delivery+CPAP, pulse volume delivery+high frequency+CPAP, and any other suitable hybrid methods and algorithms.

Thus, specific examples of pneumatic stimulation devices, systems and methods have been disclosed. The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

As used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Reference throughout this specification to “an embodiment” or “an implementation” means that a particular feature, structure, or characteristic described in connection with the embodiment is included in at least one embodiment or implementation. Thus, appearances of the phrases “in an embodiment” in various places throughout this specification are not necessarily all referring to the same embodiment or a single exclusive embodiment. Furthermore, the particular features, structures, or characteristics described herein may be combined in any suitable manner in one or more embodiments or one or more implementations.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Certain numerical values and ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

All structural and functional equivalents to the components of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A pneumatic stimulation system, comprising:
a flow device;
a patient interface fluidly coupled to the flow/pressure generation device via at least one conduit;
a controller operably connected to the flow device, and configured to receive sensor data from at least one sensor, and to control a flow of a gas from the flow device to the patient interface based at least in part on the sensor data.

2. The system of claim 1, wherein the controller is configured to control the flow of the gas in accordance with a set of control settings.

3. The system of claim 2, wherein the set of control settings is adjusted based at least in part on an artificial intelligence algorithm.

4. The system of any of claim 3, wherein the set of control settings is adjusted based on at least one of a detection of hypopnea, a detection of apnea, a degree of hypopnea, a degree of apnea, a trend based on hypopnea, a trend based on apnea, flow boost, apnea boost, base flow, or associated control settings.

5. The system of claim 2, wherein the flow device comprises at least one of a flow generation device and a pressure generation device, and at least one control setting of the set of control settings is set randomly.

6. A pneumatic stimulation system, comprising:
a flow device;
a patient interface fluidly coupled to the flow device via at least one conduit;
a controller operably connected to the flow device, and configured to cause the flow device to deliver a pulse of a gas via the patient interface.

7. The pneumatic stimulation system of claim 6, wherein the controller is configured to cause the flow device to deliver the pulse of the gas based on a time interval.

8. The pneumatic stimulation system of claim 6, wherein the flow device is a flow/pressure generation device, and wherein the controller is configured to cause the flow/pressure generation device to deliver the pulse of the gas based on a user's breathing activity.

9. A pneumatic stimulation system for a user, comprising:
a flow device;
a patient interface fluidly coupled to the flow/pressure generation device via at least one conduit;
a controller operably connected to the flow/pressure generation device, and configured to receive sensor data from at least one sensor coupled to at least one of the user, the conduit, and the flow device, and automatically adjust a control setting based at least in part on the sensor data.

10. A method, comprising providing a set of pulses of a gas to a user to pneumatically stimulate a hypoglossal nerve of the user.

11. The method of claim 10, wherein providing the set of pulses comprises providing the set of pulses via a nasal cannula.

12. The method of 11, wherein providing the set of pulses comprises providing the set of pulses via a flow generation system based on at least one control setting.

13. The method of claim 12, wherein the at least one control setting comprises at least one of a breath trigger setting, a boost flow setting, a boost flow duration setting, a base flow setting, and an auto trigger timer.

14. A pneumatic stimulation system for a user, comprising:
a flow/pressure generation device;
a patient interface fluidly coupled to the flow/pressure generation device via at least one conduit;
a controller operably connected to the flow/pressure generation device, and configured to receive sensor data from at least one sensor coupled to at least one of the user, the conduit, and the flow/pressure generation device, and determine at least one preferred control setting based at least in part on the sensor data.

15. A pneumatic stimulation system for a user, comprising:
a flow/pressure generation device;
a patient interface fluidly coupled to the flow/pressure generation device via at least one conduit;
a controller operably connected to the flow/pressure generation device, and configured to receive sensor data from at least one sensor coupled to at least one of the user, the conduit, and the flow/pressure generation device.

16. A method of determining an optimal set of control settings for pulsed gas delivery, comprising:
obtaining a first set of sensor data associated with a breathing of a person;
delivering a first set of pulses of a gas in accordance with a first set of control settings via a patient interface;
obtaining a second set of sensor data associated with the breathing of the person after delivering the first set of pulses;
delivering a second set of pulses of a gas in accordance with a second set of control settings via the patient interface; and
determining an optimal set of control settings based at least in part on at least one of the first set of sensor data and the second set of sensor data.

17. The method of claim 16, wherein determining the optimal set of control settings is based at least in part on an artificial intelligence algorithm.

18. The method of claim 16, wherein determining the optimal set of control settings is based at least in part on a machine learning algorithm.

19. The method of claim 16, wherein determining the optimal set of control settings is based at least in part on sensor data from sensors of at least two pneumatic stimulation systems.

20. A pneumatic stimulation device configured to deliver a set of pulses of a gas to a person via a patient interface in accordance with a set of control settings.

21. The device of claim 20, wherein the set of control settings are randomized.

22. The device of claim 20, wherein at least some control settings of the set of control settings are set randomly.

23. The system of any of claim 1, wherein the patient interface comprises at least one of an oral mask, a nasal pillow, a nasal mask, a full face mask, a cannula, and a total face mask.

24. The device of any of claim 20, wherein the patient interface comprises at least one of an oral mask, a nasal pillow, a nasal mask, a full face mask, a cannula, and a total face mask.

25. A system for pneumatic stimulation of a hypoglossal nerve of a person, comprising:
a flow device;
a patient interface fluidly coupled to the flow device via at least one conduit;
at least one sensor configured to detect data associated with the person's sleep;
a processing system coupled with the at least one sensor and configured to communicate the data associated with the person's sleep;
a platform, comprising:
an application communicatively coupled with a database storing trend data, and configured to receive the data associated with the person's sleep and determine an event by comparing the data associated with the person's sleep to the trend data; and
a controller operably connected to the flow device, and configured to control a delivery of a gas from the flow device to the patient interface based at least in part on the event determined by comparing the data associated with the person's sleep to the trend data.

26. The system of claim 25, wherein the flow device comprises a FT device.

27. The system of any of claim 25, wherein the flow device has an integrated humidifier.

28. The system of any of claim 25, wherein the patient interface comprises at least one of an oral mask, a nasal pillow, a nasal mask, a full face mask, a cannula, and a total face mask.

29. The system of any of claim 25, wherein the data associated with the person's sleep comprises SpO2 data.

30. The system of any of claim 25, wherein the data associated with the person's sleep comprises breath data.

31. The system of any of claim 25, wherein the at least one sensor comprises at least one of a pulse oximeter and a flow sensor.

32. The system of any of claim 25, wherein the event is at least one of apnea, hypopnea, a drop in SpO2 beyond a threshold, and a lack of patient trigger efforts for beyond a predetermined time period.

33. The system of any of claim 25, wherein the trend data comprises historic data associated with the patient's sleep.

34. The system of any of claim 25, wherein the trend data comprises current or historic data associated with an individual's sleep other than the patient.

35. The system of any of claim 25, wherein controlling the flow of the gas comprises at least one of modifying a pressure of a set of pulses of the gas delivered to the patient interface, modifying a length of pulses of the set of pulses of the gas delivered to the patient interface, and modifying a frequency of pulses of the set of pulses of the gas delivered to the patient interface.

36. The system of any of claim 25, wherein the at least one sensor comprises a flow sensor.
